(19) United States
(12) Patent Application Publication
(10) Pub. No.: US 2024/0324956 A1
(43) Pub. Date: Oct. 3, 2024

(54) DETACHABLE DETECTION DEVICE

(71) Applicant: OXTI Pte Ltd, Singapore (SG)

(72) Inventor: Chih-Feng Ho, Singapore (SG)

(21) Appl. No.: 18/127,675

(22) Filed: Mar. 29, 2023

(51) Int. Cl. A61B 5/00

(52) U.S. Cl. CPC A61B 5/681; A61B 5/742

(57) ABSTRACT

A detachable detection device includes: a first sheet and second sheet, the first sheet provided with a first side, second side and sensor, the first side a combining part, the second sheet a third side and fourth side, the third side a combined part, and the combining part in detachable connection with the combined part; and a connection part, provided with a first end and second end, the first end in connection with the second side of the first sheet, the second end the fourth side of the second sheet, and the connection part allowing the first sheet and second sheet to be bent an angle. With the above structure, the first sheet and second sheet can be bent and clamped on the wristband or watch strap with the connection part of the present invention when in use, and the combining part is then connected with the combined part.

### (a) TECHNICAL FIELD OF THE INVENTION

The present invention relates to a detection device, and more particularly to a detachable detection device.

### (b) DESCRIPTION OF THE PRIOR ART

Modern people often have the habit of wearing watches or wristbands, but if they want to detect their own physiological conditions, they need to purchase additional equipment that can monitor their physiological conditions. Taiwan Patent No. 1596455, titled “Wearable Electronic Device”, has both a watch and the function of monitoring physiological conditions for users to purchase.

If the original watch or wristband does not monitor the physiological function, it is necessary to purchase a device with this function. It is very troublesome and not beautiful to replace or wear it together with the original watch or wristband in different situations. A watch that monitors physiological functions can monitor physiological conditions, but its display position is fixed at a certain position on the strap, and it cannot follow one's own thoughts to be fixed at any position on the strap at will.

### SUMMARY OF THE INVENTION

To overcome the above disadvantages, the present invention proposes a detachable detection device, including: a first sheet and second sheet, the first sheet provided with a first side, second side and sensor, the first side a combining part, the second sheet a third side and fourth side, the third side a combined part, and the combining part in detachable connection with the combined part; and a connection part, provided with a first end and second end, the first end in connection with the second side of the first sheet, the second end the fourth side of the second sheet, and the connection part allowing the first sheet and second sheet to be bent an angle.

With the above structure, the first sheet and second sheet can be bent and clamped on the wristband or watch strap with the connection part of the present invention when in use, and the combining part is then connected with the combined part, allowing one or more of the present inventions to be fixed on any positions of the wristband or watch strap. Furthermore, only the combining part is separated from the combined part when they are not used or need to be cleaned, which can quickly remove the present invention from the wristband or watch strap.

### BRIEF DESCRIPTION OF THE DRAWINGS

FIG. 1 is a perspective view of the present invention;

FIG. 2 is a perspective view of the present invention, where a combining part is in connection with a combined part;

FIG. 3 shows an action before the use of the present invention;

FIG. 4 shows the present invention in use; and

FIG. 5 shows a plurality of devices of the present invention used together.

### DETAILED DESCRIPTION OF THE PREFERRED EMBODIMENTS

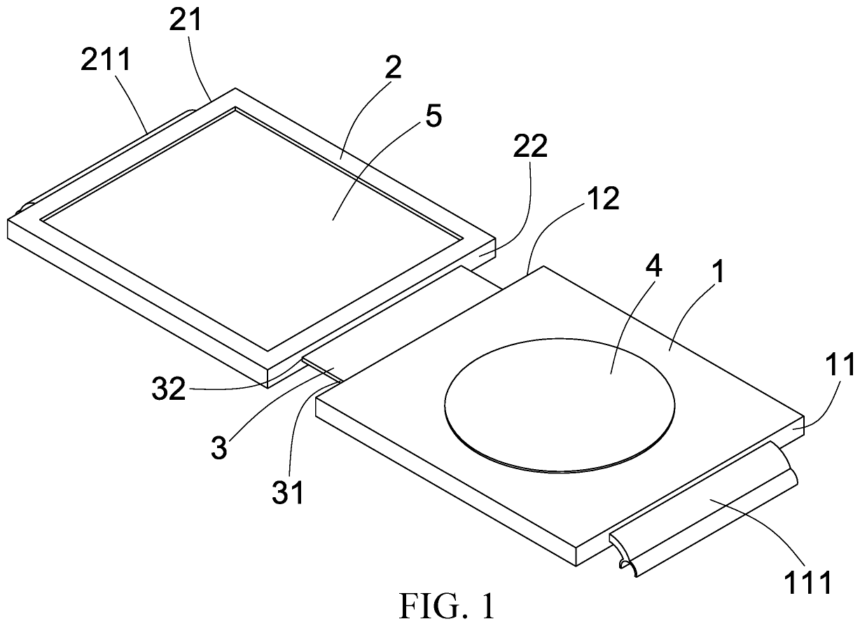

Referring to FIG. 1, a detachable detection device of the present invention includes a first sheet 1, second sheet 2 and connection part 3.

The first sheet 1 is provided with a first side 11 and second side 12, where the first side 11 is provided with a combining part 111, and the first sheet 1 a sensor 4. The second sheet 2 is provided with a third side 21 and fourth side 22, where the third side 21 is provided with a combined part 211, which is in detachable connection with the combining part 111.

The connection part 3 is provided with a first end 31 and second end 32, where the first end 31 is connected with the second side 12 of the first sheet, and the second end 32 the fourth side of the second sheet 2. Furthermore, the connection part 3 allows the first sheet 1 to be bent an angle with respect to the second sheet 2.

The sensor 4 can detect the user's physiological condition (for example, heartbeat, pulse or blood oxygen, etc.) or their location, etc., and can emit signals to the user's smart device (e.g., smart phone), allowing the user to know their physiological condition or the location.

The second sheet 2 may be provided with a display device 5, which can display the physiological condition or the location detected by the sensor 4, allowing the user to know the user's physiological condition and location by viewing the display device 5.

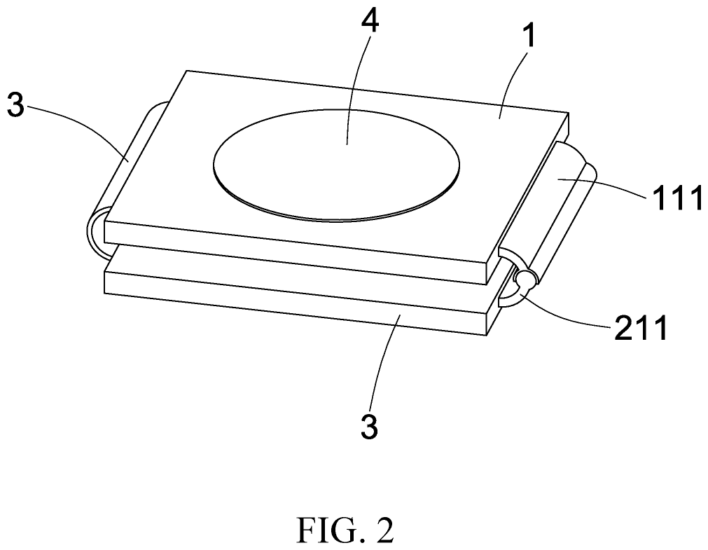

Referring to FIG. 2, the combining part 111 and combined part 211 may be connected with complementary snaps, magnetic buckles or Velcro tapes, which can be disassembled after the combining part 111 is connected with the combined part 211.

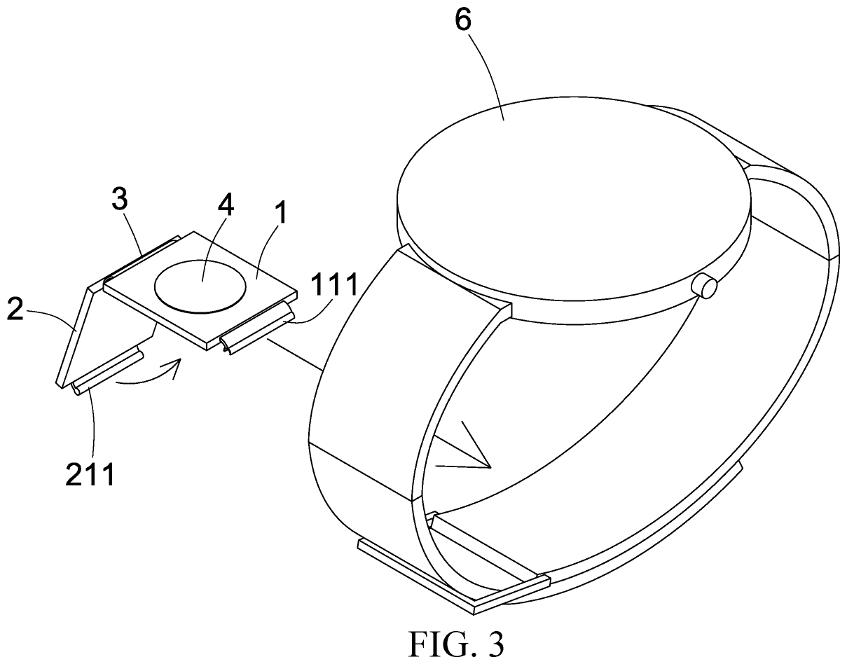

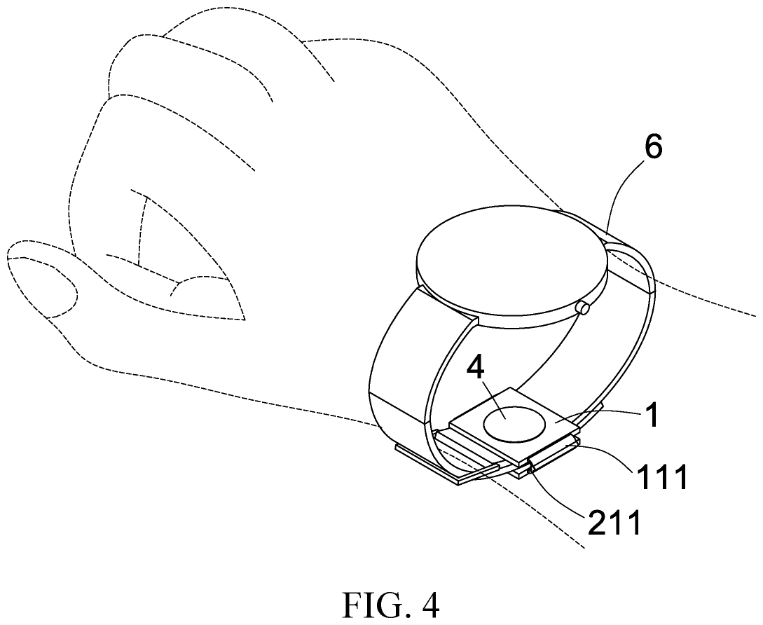

Referring to FIGS. 3 and 4, when the present invention is in use, the first sheet 1 and second sheet 2 can be bent, make it clamp its two sides on the wrist strap or watch strap 6, and the combining part 111 is then connected to the combined part 211, allowing it to be firmly fixed on any position of the wristband or watch strap 6. Furthermore, if it does not need to be used, only separating the combining part 111 from the combined part 211 can then remove the present invention from the wristband or watch strap 6 without needing to touch the wristband or watch strap 6.

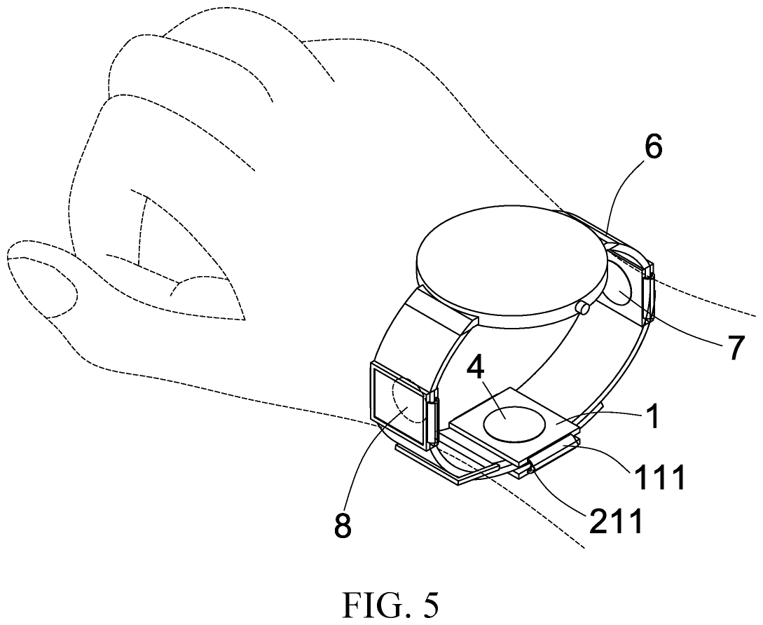

The present invention can also be used with multiple detection devices with different functions such as sensors 4, 7 and 8 as shown in FIG. 5.

The first sheet 1 and second sheet 2 can be bent and clamped on the wristband or watch strap 6 with the connection part 3 of the present invention, and the combining part 111 is then connected with the combined part 211, allowing one or more detection devices of the present invention to be fixed on any positions of the wristband or watch strap 6. Furthermore, only the combining part 111 is separated from the combined part 211 when they are not used or need to be cleaned, which can achieve the detachable effect.

I claim:

1. A detachable detection device, comprising:
a first sheet and second sheet, said first sheet provided with a first side, second side and sensor, said first side a combining part, said second sheet a third side and fourth side, said third side a combined part, and said combining part in detachable connection with said combined part; and
a connection part, provided with a first end and second end, said first end in connection with said second side of said first sheet, said second end said fourth side of said second sheet, and said connection part allowing said first sheet and second sheet to be bent an angle.

2. The detection device according to claim 1, wherein said second sheet is provided with a display device.

3. The detection device according to claim 1, wherein said combining part and combined part are complementary snaps.

4. The detection device according to claim 1, wherein said combining part and combined part are magnetic buckles.

5. The detection device according to claim 1, wherein said combining and combined part are Velcro tapes.
